SEQUENCE LISTING (58 sequences)

SEQ ID NO: 1 - protein, 15 aa, Artificial Sequence
  PD-1 Fragment
  misc_feature: Synthetic
CGAISLAPKAQIKES

SEQ ID NO: 2 - protein, 16 aa, Artificial Sequence
  PD-1 Fragment
  misc_feature: Synthetic
NWYRMSPSNQTDKLAA

SEQ ID NO: 3 - protein, 16 aa, Artificial Sequence
  PD-1 Fragment
  misc_feature: Synthetic
FRVTQLPNGRDFHMSV

SEQ ID NO: 4 - protein, 12 aa, Artificial Sequence
  PD-1 Fragment
  misc_feature: Synthetic
GWFLDSPDRPWN

SEQ ID NO: 5 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 6 - protein, 288 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 7 - protein, 288 aa, Macaca mulatta
MQIPQAPWPVVWAVLQLGWRPGWFLESPDRPWNPPTFSPALLLVTEGDNATFTCSFSNASESFVLNWYRMSPSNQTDKLAAFPEDRSQPGRDCRFRVTQLPNGRDFHMSVVRARRNDSGTYLCGAISLAPKAQIKESLRAELRVTERRAEVPTAHPSPSPRPAGQFQALVVGVVGGLLGSLVLLVWVLAVICSRAAQGTIEARRTGQPLKEDPSAVPVFSVDYGELDFQWREKTPEPPAPCVPEQTEYATIVFPSGLGTSSPARRGSADGPRSPRPLRPEDGHCSWPL

SEQ ID NO: 8 - protein, 286 aa, Equus caballus
MGTLRAPWPFVWVLLQLGWLPAWLLDSPDRPWRPLTFSPARLMVPEGANATFTCSFSNTSEHFVLNWYRMSPSNQTDKLAAFPEDSSQPGRSGRFRVTRLPNGRDFHMSVLAARRNDSGIYLCGAISLPPKTQINESPRAELTVTERIPEPPTEHPSPPPSPAGQLQGLVVGITSLLVGVLLVLLLACVLTTTFPRAARGACGRSGDEPLEGPSAPPAFSVDYGELDFQWREKTPEPPAPCVPEQTEYATIVFPGRPGSQGRRASADDPQGPRPLRPEDGHCSWPL

SEQ ID NO: 9 - protein, 287 aa, Equus caballus
MGTLRAPWPFVWVLLQLGWLPAWLLDSPDRPWRPLTFSPARLMVPEGANATFTCSFSNTSEHFVLNWYRMSPSNQTDKLAAFPEDSSQPGRSGRFRVTRLPNGRDFHMSVLAARRNDSGIYLCGAISLPPKTQINESPRAELTVTERIPEPPTEHPSPPPSPAGQLQGLVVGITSLLVGVLLVLLLACVLTTTFPRAARGACGRSGDEPLKEGPSAPPAFSVDYGELDFQWREKTPEPPAPCVPEQTEYATIVFPGRPGSQGRRASADDPQGPRPLRPEDGHCSWPL

SEQ ID NO: 10 - protein, 288 aa, Canis familiaris
MGSRRGPWPLVWAVLQLGWWPGWLLDSPDRPWSPLTFSPAQLTVQEGENATFTCSLADIPDSFVLNWYRLSPRNQTDKLAAFQEDRIEPGRDRRFRVTRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 11 - protein, 278 aa, Felis catus
MGTPRAPWPLVWAVLQLGWWPGWLLDSPYRPWSPLTFSPAQLTVLEGENATFVCHLPDVPESFVLNWYRVSPRNQTDKLAAFQENHTEPGKDRRFRVTRLPSGQDFHTTILAAQLNDSGIYLCGAIYLPPNTQIYESPRAELTVKERVLEPPTESPSPPPRLTGQGQGLVVGVTSVLVGVLLLLLLTWVLAAAFPRATRGACACGSEDEPLKEGPSAAPVFTVDYGELDFQWREKTPEPPAPCAPEQTEYATIVFPSRPGSPGPLPLRPEDGPCPWPL

SEQ ID NO: 12 - protein, 282 aa, Bos taurus
MGTPRALWPLVWAVLQLGCWPGWLLEASSRPWSALTFSPPRLVVPEGANATFTCSFSSKPERFVLNWYRKSPSNQMDKLAAFPEDRSQPSRDRRFRVTPLPDGQQFNMSIVAAQRNDSGVYFCGAIYLPPRTQINESHSAELMVTEAVLEPPTEPPSPQPRPEGQMQSLVIGVTSVLLGVLLLPPLIWVLAAVFLRATRGGCARRSQDQPPKEGCPSVPAVTVDYGELDFQWREKTPEPAAPCVPEQTEYATIVFPGRRASADSPQGPWPLRTEDGHCSWPL

SEQ ID NO: 13 - protein, 282 aa, Bos taurus; identical to SEQ ID NO: 12

SEQ ID NO: 14 - protein, 282 aa, Bos taurus; identical to SEQ ID NO: 12

SEQ ID NO: 15 - protein, 288 aa, Sus scrofa
MGTPRALWPVVWVVLQLRWWPGWLLDAPSRPRGPLTLSPAQLTVPEGANATFTCSFPSEPKHFILNWYRLSPSNQTDKLAAFSEDGSQPGRDPRFHVTPLPNGRDFHMSVVATRRNDSGTYFCGAIYLPPKTQINESHQAKLTVTERVLELPTEHPSCPPRPEGHLEGQVLVITSVLLGLLLLLLLAWSLAAFFLWAPRGDRAHRTENQPRKEGASSGLVFTVDYGELDFQWREKTPVPSAACVSEQTEYATIVFPERPGSPGRRASADSPQGPWPQRTEDGHCSWPL

SEQ ID NO: 16 - protein, 287 aa, Sus scrofa
MGTPRALWPVVWVVLQLRWWPGWLLDAPSRPRGPLTLSPAQLTVPEGANATFTCSFPSEPKHFILNWYRLSPSNQTDKLAAFSEEGSQPGRDPRFHVTPLPNGRDFHMSVVATRRNDSGTYFCGAIYLPPKTQINESHQAKLTVTERVLELPTEHPSCPPRPEGHLEGQVLVITSVLLGLLLLLLLAWSLAAFFLWAPRGDRAHRRENQPREGASSGLVFTVDYGELDFQWREKTPVPSAACVSEQTEYATIVFPERPGSPGRRASADSPQGPWPQRTEDGHCSWPL

SEQ ID NO: 17 - protein, 284 aa, Ovis aries
MGTPRAPWPLVWAVLQLGCWPGWLLEASSRPWSALTFSPARLVVPEGANATFTCSFSSKPEEHFVLNWYRMSPSNQTDKLAAFPEDRSQPGRDRRFRVTPLPGGQQFHMSIVAAQRNDSGAYFCGAIYLPPRTQINESHPAELRVTEGVLEPPTEHPSPQPRPEGQMQSLVIGVTSFILGVLLLLLLIWVLAAVFLGATRGDGSPKALPLAPSLASCPNPIPSVSPIREDLDWASRAGGPLGNPSAGPALGERGREDGPDHGRHPGWTSARALSSCNSQGTELL

SEQ ID NO: 18 - protein, 283 aa, Capra hircus
MGTPRAPWPLVWAVLQLGCWPGWLLEASSRPWSALTFSPARLVVSEGANATFTCSFSSKPEEHFVLNWYRMSPSNQTDKLAAFPEDRSQPGRGRRFRVTPLPGGRQFHMSIVAAQRDDSGAYFCGAIYLPPRTQINESRPAELRVTEEVLEPPTEHPSPQPRPEGQMQSLVIGVTSFILGVLLLLLLIWVLAAAFLGATRGGCACRSRDQLPKEGSPSVPAVTVDYGELDFQWREKTPEPAAPCVPEQTEYATIVFPGRRASADSPQGPWPLRTEDGHCSWPL

SEQ ID NO: 19 - protein, 282 aa, Capra hircus
MGTPRAPWPLVWAVLQLGCWPGWLLEASSRPWSALTFSPARLVVSEGANATFTCSFSSKPEEHFVLNWYRMSPSNQTDKLAAFPEDRSQPGRGRRFRVTPLPGGRQFHMSIVAAQRDDSGAYFCGAIYLPPRTQINESRPAELRVTEEVLEPPTEHPSPQPRPEGQMQSLVIGVTSFILGVLLLLLLIWVLAAAFLGATRGGCACRSRDQLPEGSPSVPAVTVDYGELDFQWREKTPEPAAPCVPEQTEYATIVFPGRRASADSPQGPWPLRTEDGHCSWPL

SEQ ID NO: 20 - protein, 290 aa, Camelus bactrianus
MGTPQALWPLVWAVLQLGWRPGWLLDSPSRPWSPLTFSPARLTVPEGANATFTCSFSSKPEHFLLNWYRMSPSNQTDKLAAFPEDRSQPARDRRFRVTPLPNGRDFHMSVIAAQRNDSGVYFCGAIYLPPKTQINESHPAELTVTERVLELSPTERPSSPPRTEGQLQGLVIGITSVLLGVLLLLLLIWVLAAVFLGATRGACTRRSEDSEPRKEGSTAAPVFTVDYGELDFQWREKTPEPSAPCVPEQTEYATIVFPGRPGSPGRRASADSLQGPRSLRTEDGHCSWPL

SEQ ID NO: 21 - protein, 289 aa, Camelus bactrianus
MGTPQALWPLVWAVLQLGWRPGWLLDSPSRPWSPLTFSPARLTVPEGANATFTCSFSSKPEHFLLNWYRMSPSNQTDKLAAFPEDRSQPARDRRFRVTPLPNGRDFHMSVIAAQRNDSGVYFCGAIYLPPKTQINESHPAELTVTERVLELSPTERPSSPPRTEGQLQGLVIGITSVLLGVLLLLLLIWVLAAVFLGATRGACTRRSEDSEPREGSTAAPVFTVDYGELDFQWREKTPEPSAPCVPEQTEYATIVFPGRPGSPGRRASADSLQGPRSLRTEDGHCSWPL

SEQ ID NO: 22 - protein, 290 aa, Camelus ferus; identical to SEQ ID NO: 20

SEQ ID NO: 23 - protein, 289 aa, Camelus ferus; identical to SEQ ID NO: 21

SEQ ID NO: 24 - protein, 290 aa, Camelus dromedarius
MGTPQALWPLVWAVLQLGWRPGWLLDSPSRPWSPLTFSPARLTVPEGANATFTCSFSSKPEHFLLNWYRMSPSNQTDKLAAFPEDRSQPARDRRFRVTPLPNGRDFHMSVIAAQRNDSGVYFCGAIYLPPKTQINESHPAELTVTERVLELSPTERPSSPPRTEGQLQGLVIGITSVLLGVLLLLLLIWVLAAVFLGATRGACTRRSEDSEPREGSTAAPVFTVDYGELDFQWREKTPEPSAPCVPEQTEEYATIVFPGRPGSPGRRASADSLQGPRSLRTEDGHCSWPL

SEQ ID NO: 25 - protein, 289 aa, Camelus dromedarius; identical to SEQ ID NO: 21

SEQ ID NO: 26 - protein, 16 aa, Artificial Sequence
  Immunogenic polypeptide
  misc_feature: Synthetic
SGAISLAPKAQIKESC

SEQ ID NO: 27 - protein, 16 aa, Artificial Sequence
  Immunogenic polypeptide
  misc_feature: Synthetic
NWYRMSPSNQTDKLAC

SEQ ID NO: 28 - protein, 16 aa, Artificial Sequence
  Immunogenic polypeptide
  misc_feature: Synthetic
CNWYRMSPSNQTDKLA

SEQ ID NO: 29 - protein, 17 aa, Artificial Sequence
  Immunogenic polypeptide
  misc_feature: Synthetic
CFRVTQLPNGRDFHMSV

SEQ ID NO: 30 - protein, 17 aa, Artificial Sequence
  Immunogenic polypeptide
  misc_feature: Synthetic
FRVTQLPNGRDFHMSVC

SEQ ID NO: 31 - protein, 16 aa, Artificial Sequence
  Immunogenic polypeptide
  misc_feature: Synthetic
CQGAISLAPKAQIKEK

SEQ ID NO: 32 - protein, 15 aa, Artificial Sequence
  Immunogenic polypeptide
  misc_feature: Synthetic
QAISLAPKAQIKEKC

SEQ ID NO: 33 - protein, 16 aa, Artificial Sequence
  Immunogenic polypeptide
  misc_feature: Synthetic
CQWYRMSPSNQTDKLK

SEQ ID NO: 34 - protein, 16 aa, Artificial Sequence
  Immunogenic polypeptide
  misc_feature: Synthetic
QWYRMSPSNQTDKLKC

SEQ ID NO: 35 - protein, 19 aa, Artificial Sequence
  Immunogenic polypeptide
  misc_feature: Synthetic
CQFRVTQLPNGRDFHMSVK

SEQ ID NO: 36 - protein, 19 aa, Artificial Sequence
  Immunogenic polypeptide
  misc_feature: Synthetic
QFRVTQLPNGRDFHMSVKC

SEQ ID NO: 37 - protein, 16 aa, Artificial Sequence
  Immunogenic polypeptide
  misc_feature: Synthetic
  MISC_FEATURE (15)..(15): Norleucine (Nle)
CAISLAPKAQIKECXC

SEQ ID NO: 38 - protein, 17 aa, Artificial Sequence
  Immunogenic polypeptide
  misc_feature: Synthetic
  MISC_FEATURE (16)..(16): Norleucine (Nle)
CWYRMSPSNQTDKLCXC

SEQ ID NO: 39 - protein, 20 aa, Artificial Sequence
  Immunogenic polypeptide
  misc_feature: Synthetic
  MISC_FEATURE (19)..(19): Norleucine (Nle)
CFRVTQLPNGRDFHMSVCXC

SEQ ID NO: 40 - protein, 19 aa, Artificial Sequence
  Immunogenic polypeptide
  misc_feature: Synthetic
  MISC_FEATURE (17)..(17): Norleucine (Nle)
CGAISLAPKAQIKECGXEC

SEQ ID NO: 41 - protein, 18 aa, Artificial Sequence
  Immunogenic polypeptide
  misc_feature: Synthetic
  MISC_FEATURE (15)..(15): Norleucine (Nle)
CAISLAPKAQIKCGXEEC

SEQ ID NO: 42 - protein, 21 aa, Artificial Sequence
  Immunogenic polypeptide
  misc_feature: Synthetic
  MISC_FEATURE (19)..(19): Norleucine (Nle)
CWYRMSPSNQTDKLACGEXEC

SEQ ID NO: 43 - protein, 21 aa, Artificial Sequence
  Immunogenic polypeptide
  misc_feature: Synthetic
  MISC_FEATURE (19)..(19): Norleucine (Nle)
CRVTQLPNGRDFHMSCEEXGC

SEQ ID NO: 44 - protein, 14 aa, Artificial Sequence
  Immunogenic polypeptide
  misc_feature: Synthetic
CGWFLDSPDRPWNE

SEQ ID NO: 45 - protein, 14 aa, Artificial Sequence
  Immunogenic polypeptide
  misc_feature: Synthetic
GWFLDSPDRPWNEC

SEQ ID NO: 46 - protein, 14 aa, Artificial Sequence
  Immunogenic polypeptide
  misc_feature: Synthetic
GWFLDSPDRPWNEC

SEQ ID NO: 47 - protein, 16 aa, Artificial Sequence
  Immunogenic polypeptide
  misc_feature: Synthetic
QGWFLDSPDRPWNEKC

SEQ ID NO: 48 - protein, 16 aa, Artificial Sequence
  Immunogenic polypeptide
  misc_feature: Synthetic
CQGWFLDSPDRPWNEK

SEQ ID NO: 49 - protein, 18 aa, Artificial Sequence
  Immunogenic polypeptide
  misc_feature: Synthetic
  MISC_FEATURE (16)..(16): Norleucine (Nle)
CGWFLDSPDRPWNCEXEC

SEQ ID NO: 50 - protein, 14 aa, Artificial Sequence
  PD-1 Fragment
  misc_feature: Synthetic
GAISLAPKAQIKES

SEQ ID NO: 51 - protein, 13 aa, Artificial Sequence
  PD-1 Fragment
  misc_feature: Synthetic
GAISLAPKAQIKE

SEQ ID NO: 52 - protein, 12 aa, Artificial Sequence
  PD-1 Fragment
  misc_feature: Synthetic
AISLAPKAQIKE

SEQ ID NO: 53 - protein, 13 aa, Artificial Sequence
  PD-1 Fragment
  misc_feature: Synthetic
CGAISLAPKAQIK

SEQ ID NO: 54 - protein, 11 aa, Artificial Sequence
  PD-1 Fragment
  misc_feature: Synthetic
AISLAPKAQIK

SEQ ID NO: 55 - protein, 15 aa, Artificial Sequence
  PD-1 Fragment
  misc_feature: Synthetic
NWYRMSPSNQTDKLA

SEQ ID NO: 56 - protein, 13 aa, Artificial Sequence
  PD-1 Fragment
  misc_feature: Synthetic
WYRMSPSNQTDKL

SEQ ID NO: 57 - protein, 14 aa, Artificial Sequence
  PD-1 Fragment
  misc_feature: Synthetic
WYRMSPSNQTDKLA

SEQ ID NO: 58 - protein, 14 aa, Artificial Sequence
  PD-1 Fragment
  misc_feature: Synthetic
RVTQLPNGRDFHMS

(19) United States
(12) Patent Application Publication
(10) Pub. No.: US 2021/0371496 A1
(43) Pub. Date: Dec. 2, 2021

(54) ANTI-PD-1 VACCINE COMPOSITION

(71) Applicants: PEPTINOV SAS, PARIS (FR); CONSERVATOIRE NATIONAL DES ARTS ET METIERS, PARIS (FR)

(72) Inventors: Lucille DESALLAIS, DEUIL LA BARRE (FR); Jean-Pierre SALLES, EGUILLES (FR); Jean-François ZAGURY, LE VESINET (FR)

(21) Appl. No.: 16/978,919

(22) PCT Filed: Mar. 5, 2019
(86) PCT No.: PCT/EP2019/055461

§ 371 Date: Jun. 14, 2021

(30) Foreign Application Priority Data
Mar. 5, 2018 (FR) 1851852

(51) Int. Cl. C07K 14/705; C07K 16/28

(52) U.S. Cl. CPC C07K 14/70521; A61K 39/00; C07K 16/2827

(57) ABSTRACT

The present invention relates to a polypeptide which comprises or consists of:—a first sequence consisting of at least 8 contiguous amino acid residues selected from within the sequence extending from amino acid residues 123 to 137 of the PD-1 protein, and at most 30 contiguous amino acid residues selected from within the complete sequence of the PD-1 protein; and/or—a second sequence consisting of at least 8 contiguous amino acid residues selected from within the sequence extending from amino acid residues 66 to 81 of the PD-1 protein, and at most 30 contiguous amino acid residues selected from within the complete sequence of the PD-1 protein; and/or—a third sequence consisting of at least 8 contiguous amino acid residues selected from within the sequence extending from amino acid residues 95 to 110 of the PD-1 protein, and at most 30 contiguous amino acid residues selected from within the complete sequence of the PD-1 protein; and/or—a fourth sequence consisting of at least 8 contiguous amino acid residues selected from within the sequence extending from amino acid residues 22 to 33 of the PD-1 protein, and at most 30 contiguous amino acid residues selected from within the complete sequence of the PD-1 protein.

The Sequence Listing in ASCII text file format of 62,787 bytes in size, created on Jun. 14, 2021, with the file name “2021-06-14Sequence_listing-DESALLAIS3,” filed in the U.S. Patent and Trademark Office on Jun. 14, 2021, is hereby incorporated herein by reference.

### FIELD OF THE INVENTION

This invention relates to polypeptides useful in eliciting an immune response directed against the PD-1 protein.

### TECHNICAL BACKGROUND

It has now been established that the intensity of the natural immune response against cancer antigens is correlated with better prognoses for patients with several types of neoplasia. Clinical observations, supported by extensive experimental evidence, have helped define the concept of cancer immunosurveillance, whereby emerging tumors are usually eradicated by the immune system, except in circumstances where cancer cells have evolved to escape immune detection.

Cancer immunotherapy—a direct application of the concept of immunosurveillance—which has seen spectacular growth and success over the past decade, has revolutionized the clinical management of a wide range of malignant tumors previously associated with poor prognosis.

At the forefront of the development of immunotherapy are monoclonal antibodies, immune checkpoint blockers (ICB), which have been known to have great success in oncology thanks to their broad activity on several types of tumors, the durability of their responses and their capacity for treatment of metastatic chemo-resistant tumors.

Of the checkpoint blocking strategies, the two most important (in terms of clinical success to date) are the targeting by specific monoclonal antibodies of cytotoxic T cell associated protein 4 (CTLA-4) and of the interaction between the programmed cell death protein 1 (PD-1) and the ligand of this programmed cell death protein 1 (PD-L1). In particular, inhibition of PD-L1 signaling has been proposed as a means of improving T cell immunity for the treatment of a cancer (anti-tumor immunity), but also in the treatment of infections (acute and chronic persistent infection).

To date, four ICBs targeting the PD-1/PD-L1 axis were thus notably approved by the U.S. Food and Drug Administration (FDA) (for a review, see for example, Abdin et al. (2018) Cancers 10 32):
(1) pembrolizumab, an anti-PD-1 monoclonal antibody (mAb) approved for patients with metastatic unresectable melanoma or advanced metastatic non-small cell lung carcinoma (NSCLC) whose tumors express PD-L1;
(2) nivolumab, an anti-PD-1 monoclonal antibody (mAb) approved for unresectable or metastatic melanoma, advanced metastatic NSCLC progressing with or after platinum-based chemotherapy, and advanced renal carcinoma, including metastatic;
(3) arezolizumab, a recently approved anti-PD-L1 monoclonal antibody (mAb) for the treatment of locally advanced or metastatic urothelial carcinoma unresponsive to platinum-based chemotherapy;
(4) avelumab, an anti-PD-L1 monoclonal antibody (mAb) recently approved for the treatment of metastatic Merkel cell carcinoma.

In addition, these ICBs already approved for a number of indications could also be recognized in the future as useful in the treatment of other forms of cancers.

Furthermore, ICBs targeting PD-L1 have been shown to be very effective in melanoma, NSCLC and renal carcinoma.

However, all the products marketed or developed as ICBs targeting the PD-1/PD-L1 axis are monoclonal antibodies and are therefore affected by the same limitations as other monoclonal antibody treatments: high cost, the need for frequent re-administration and the development of an immune reaction directed against the administered monoclonal antibodies.

It is therefore a subject-matter of this invention to overcome these drawbacks.

### SUMMARY OF THE INVENTION

This invention arises from the unexpected demonstration, by the inventors, that polypeptides derived from sequences spanning amino-acid residues 22 to 33, 66 to 81, 95 to 110, and 123 to 137 of human PD-1 protein made the production of antibodies neutralizing the PD-1 protein possible in mice to which they were administered.

This invention therefore relates to a polypeptide comprising, or consisting of:
a first sequence consisting of at least 8 contiguous amino-acid residues selected from within the sequence spanning amino-acid residues 123 to 137 of the PD-1 protein and at most of 30 contiguous amino-acid residues selected from the complete sequence of the PD-1 protein, or a variant sequence having at least 75% identity with the first sequence; and/or
a second sequence consisting of at least 8 contiguous amino-acid residues selected from within the sequence spanning amino-acid residues 66 to 81 of the PD-1 protein and at most of 30 contiguous amino-acid residues selected from the complete sequence of the PD-1 protein, or a variant sequence having at least 75% identity with the second sequence; and/or
a third sequence consisting of at least 8 contiguous amino-acid residues selected from within the sequence spanning amino-acid residues 95 to 110 of the PD-1 protein and at most of 30 contiguous amino-acid residues selected from the complete sequence of the PD-1 protein, or a variant sequence having at least 75% identity with the third sequence; and/or
a fourth sequence consisting of at least 8 contiguous amino-acid residues selected from within the sequence spanning amino-acid residues 22 to 33 of the PD-1 protein and at most f30 contiguous amino-acid residues selected from the complete sequence of the PD-1 protein, or a variant sequence having at least 75% identity with the fourth sequence;
   provided that the polypeptide is different from the PD-1 protein and that it does not consist of a portion of more than 30 contiguous amino-acid residues of the PD-1 protein, and provided that polypeptides consisting of variant sequences of the first, second, third and fourth sequences, respectively, make it possible to elicit an immune response directed against the PD-1 protein.

In a preferred embodiment, the invention relates more particularly to a polypeptide comprising, or consisting of:
a first sequence consisting of SEQ ID NO: 1, 50, 51, 52, 53, or 54, or a variant sequence having at least 75% identity with the first sequence; and/or
a second sequence consisting of SEQ ID NO: 2, 55, 56 or 57, or a variant sequence having at least 75% identity with the second sequence; and/or
a third sequence consisting of SEQ ID NO: 3 or 58, or a variant sequence having at least 75% identity with the third sequence; and/or
a fourth sequence consisting of SEQ ID NO: 4, or a variant sequence having at least 75% identity with the fourth sequence;
   provided that polypeptides consisting of variant sequences of the first, second, third, and fourth sequences, respectively, make it possible to elicit an immune response directed against the PD-1 protein.

This invention relates furthermore to a nucleic acid encoding a polypeptide as defined above, or the complement thereof.

This invention relates furthermore to:
at least one polypeptide, as defined above, or
at least one nucleic acid, as defined above, for use as a medicament, in particular a vaccine. In one particular embodiment of the invention, the medicament, in particular the vaccine, as defined above also comprises at least one other compound intended for the prevention or treatment of a disease linked to or due to the expression of the PD-1 protein or of the PD-L1 protein, of a cancer, or of an infectious disease.

This invention also relates to a pharmaceutical composition, in particular a vaccine, comprising, as an active ingredient:
at least one polypeptide as defined above, or
at least one nucleic acid as defined above, optionally in combination with at least one pharmaceutically acceptable vehicle. In one particular embodiment of the invention, the pharmaceutical composition, in particular, a vaccine, as defined above, also comprises at least one other compound to be used for the prevention or treatment of a disease linked to or due to the expression of the PD-1 protein or of the PD-L1 protein, of cancer or of an infectious disease.

This invention furthermore relates to the use of a polypeptide, as defined above, for the preparation of an antibody, an antibody fragment or an aptamer.

This invention furthermore relates to a method for preparing an antibody, an antibody fragment or an aptamer comprising the step of administering a polypeptide, as defined above, to an organism that produces antibodies or the step of selecting by affinity an antibody, an antibody fragment or an aptamer, which binds to the polypeptide, as defined above.

This invention also relates to an anti-PD-1 antibody, antibody fragment, or aptamer specifically directed against the polypeptide as defined above, provided that the polypeptide does not comprise more than two amino-acid residues in addition to the first, second, third, or fourth sequence or their respective variant sequences.

This invention furthermore relates to an antibody, an antibody fragment, or an aptamer, as defined above, for use as a medicament. In one particular embodiment of the invention, the medicament as defined above also comprises at least one other compound to be used for the prevention or treatment of a disease linked to or due to the expression of the PD-1 protein or of the PD-L1 protein, of a cancer or of an infectious disease.

This invention also relates to a pharmaceutical composition comprising, as an active ingredient, an antibody, an antibody fragment, or an aptamer as defined above, optionally in combination with a pharmaceutically acceptable vehicle. In one particular embodiment of the invention, the pharmaceutical composition as defined above also comprises at least one other compound to be used for the prevention or treatment of a disease linked to or due to the expression of the PD-1 protein or of the PD-L1 protein, of a cancer or of an infectious disease.

This invention furthermore relates to a polypeptide as defined above, a nucleic acid as defined above, or a pharmaceutical composition as defined above, for use in a method for eliciting an immune response directed against the PD-1 protein in a subject. In one particular embodiment of the invention, the polypeptide, the nucleic acid or the pharmaceutical composition is used in combination with at least one other compound useful for eliciting an immune response directed against the PD-1 protein.

This invention furthermore relates to a method for eliciting an immune response directed against the PD-1 protein in a subject, comprising administering to the subject an effective amount of a polypeptide, as defined above, of a nucleic acid, as defined above, or of a pharmaceutical composition, as defined above. In one particular embodiment of the invention, the polypeptide, the nucleic acid or the pharmaceutical composition is administered in combination with at least one other compound useful for eliciting an immune response directed against the PD-1 protein.

This invention furthermore relates to the use of a polypeptide as defined above or a nucleic acid, as defined above, for the preparation of a medicament intended to elicit an immune response directed against the PD-1 protein in a subject. In one particular embodiment of the invention, the medicament also comprises at least one other compound useful for eliciting an immune response directed against the PD-1 protein.

This invention furthermore relates to a polypeptide as defined above, a nucleic acid as defined above, a pharmaceutical composition as defined above, or an antibody, an antibody fragment or an aptamer as defined above, for use in a method of preventing or treating a disease linked to or due to the PD-1 protein in a subject. In one particular embodiment of the invention, the polypeptide, nucleic acid, pharmaceutical composition, or the antibody, the antibody fragment or the aptamer is used in combination with at least one other therapy to be used for the prevention or treatment of a disease linked to the PD-1 protein, of a cancer or of an infectious disease.

This invention furthermore relates to a method of preventing or treating a disease linked to or due to the expression of the PD-1 protein or of the PD-L1 protein in a subject, comprising the administration to the subject of an effective amount of a polypeptide, as defined above, of a nucleic acid as defined above, of a pharmaceutical composition as defined above, or of an antibody, of an antibody fragment or of an aptamer as defined above. In a particular embodiment of the invention, the method comprises at least one other therapy intended for the prevention or treatment of a disease linked to the expression of the PD-1 protein or of the PD-L1 protein, of a cancer, or of an infectious disease.

This invention furthermore relates to the use of a polypeptide as defined above, of a nucleic acid as defined above, or of an antibody, of an antibody fragment or of an aptamer as defined above, for the preparation of a medicament intended for the prevention or treatment of a disease linked to or due to the expression of the PD-1 protein or the PD-L1 protein in a subject. In one particular embodiment of the invention, the medicament comprises at least one other compound intended for the prevention of a disease linked to or due to the expression of the PD-1 protein or the PD-L1 protein, of a cancer or of an infectious disease.

This invention furthermore relates to products containing:
a polypeptide or a nucleic acid as defined above, and
at least one other compound to be used for the prevention or treatment of a disease linked to or due to the expression of the PD-1 protein or of the PD-L1 protein, of a cancer or of an infectious disease,
   as a combination product for simultaneous, separate or sequential use for the prevention or treatment of a disease linked to or due to the expression of the PD-1 protein or of the PD-L1 protein in a subject, of a cancer or of an infectious disease.

### DESCRIPTION OF THE INVENTION

As a preliminary matter, it should be noted that the term “comprising” means “including,” “containing” or “encompassing,” that is to say that when an object “comprises” an element or several elements, elements other than those mentioned may also be included in the object. In contrast, the expression “consisting of” means “constituted by,” that is to say, that when an object “consists of” an element or several elements, the object cannot include elements other than those mentioned.

### Polypeptide

### Definition of the PD-1 Protein

The PD-1 protein is programmed cell death protein 1, also called PDC1 or even cluster of differentiation 279, CD 279, it is well known to those skilled in the art.

### Preferred Species and Sequences of the PD-1 Protein

Preferably, the PD-1 protein according to the invention is selected from the group consisting of the human PD-1 protein, the mouse PD-1 protein, the monkey PD-1 protein, the horse PD-1 protein, the bovine PD-1 protein, the pig PD-1 protein, the sheep PD-1 protein, the goat PD-1 protein, the camel PD-1 protein, in particular of wild or domestic camel, the dromedary PD-1 protein, the dog PD-1 protein, and the cat PD-1 protein. Particularly preferably, the PD-1 protein is the human PD-1 protein.

Preferably:
the human PD-1 protein (hPD-1) is as described in the UniProt/SwissProt database under reference Q15116 and consists of SEQ ID NO: 5,
the mouse PD-1 protein (mPD-1) is as described in the UniProt/SwissProt database under reference Q02242 and consists of SEQ ID NO: 6,
the monkey PD-1 protein is as described in the Genbank database under reference NP_001107830 and consists of SEQ ID NO: 7,
the horse PD-1 protein is as described in the Genbank database under reference XP_0234498583.1 or XP_023498582.1 and consists of SEQ ID NO: 8 or 9,
the dog PD-1 protein is as described in the SwissProt database under reference A0A024FCJ9 and consists of SEQ ID NO: 10,
the cat PD-1 protein is as described in the Genbank database under reference NP_001138982.1 and consists of SEQ ID NO: 11.
the bovine PD-1 protein is as described in the Genbank database under reference BAX73992.1; DAA30855.1 or NP_001076975.1 and consists of SEQ ID NO: 12 or 13 or 14,
the pig PD-1 is as described in the Genbank database under reference NP_001191308.1 or XP_020930289.1 and consists of SEQ ID NO: 15 or 16,
the sheep PD-1 protein is as described in the Genbank database under reference XP_012031617.1 and consists of SEQ ID NO: 17.
the goat PD-1 protein is as described in the Genbank database under reference XP_013818552.2 or XP_017899964.1 and consists of SEQ ID NO: 18 or 19.
the domestic camel PD-1 protein is as described in the Genbank database under reference XP_010946391.1 or XP_010946392.1 and consists of SEQ ID NO: 20 or 21.
the wild camel PD-1 protein is as described in the Genbank database under the reference XP_014412330.1 or XP_014412331.1 and consists of SEQ ID NO: 22 or 23.
The dromedary PD-1 protein is as described in the Genbank database under reference XP_010986792.1 or XP_010986793.1 and consists of SEQ ID NO: 24 or 25.

### Numbering of the Amino-Acid Residues

As understood herein, the amino-acid residue numbering of the PD-1 protein begins with the first amino-acid residue, usually a methionine (M), forming the N-terminus of the full PD-1 encoded by the open reading frame of the PD-1 gene, that is to say including its signal peptide. Furthermore, the numbering of the amino-acid residues of the PD-1 protein used herein is defined with reference to the human PD-1 protein. It is thus easy for those skilled in the art to determine the amino-acid residue of a PD-1 protein corresponding to a position number to which reference is made according to the invention: it suffices to align the sequence of the PD-1 protein for which it is desired to determine the amino-acid residue corresponding to a position number with a sequence of the human PD-1 protein, in particular SEQ ID NO: 5, so as to optimize the percentage identity between the two aligned sequences, then identify the amino-acid residue corresponding to the sought position number, as being that which is aligned with the amino-acid residue of the sequence of the human PD-1 protein, which bears this position number.

### First, Second, Third, and Fourth Sequences

Preferably, the first sequence, the second sequence, the third sequence, and the fourth sequence consist of at least 8, 9, 10, 11, 12 contiguous amino-acid residues selected respectively from within the sequence spanning amino-acid residues 123 to 137, 66 to 81, and 95 to 110 of the PD-1 protein or consist of at least the sequence spanning the amino-acid residues 123 to 137, 66 to 81, 95 to 110, and 22 to 33, respectively, of the PD-1 protein.

Also preferably, the first sequence, the second sequence, the third sequence, and the fourth sequence according to the invention consist respectively of at most 29, 28, 27, 26, 25, 24, 23, 22, 21, 20 contiguous amino-acid residues selected from the complete sequence of the PD-1 protein, or respectively consist of at most amino-acid residues 123 to 137, 66 to 81, 95 to 110, and 22 to 33 of the PD-1 protein.

Preferably:
the first sequence according to the invention consists of SEQ ID NO: 1, 50, 51, 52, 53 or 54,
the second sequence according to the invention consists of SEQ ID NO: 2, 55, 56 or 57,
the third sequence according to the invention consists of SEQ ID NO: 3 or 58, and
the fourth sequence consists of SEQ ID NO: 4.

The human PD-1 protein residues corresponding to these sequences are shown in the table below:

| | SEQ | hPD-1 |
|---|---|---|
| | ID | amino-acid |
| Sequence | NO: | residues |
| CGAISLAPKAQIKES | 1 | 123-137 |
| GAISLAPKAQIKES | 50 | 124-137 |
| GAISLAPKAQIKE | 51 | 124-136 |
| AISLAPKAQIKE | 52 | 125-136 |
| CGAISLAPKAQIK | 53 | 123-135 |
| AISLAPKAQIK | 54 | 125-135 |
| NWYRMSPSNQTDKLAA | 2 | 66-81 |
| NWYRMSPSNQTDKLA | 55 | 66-80 |
| WYRMSPSNQTDKL | 56 | 67-79 |
| WYRMSPSNQTDKLA | 57 | 67-80 |
| FRVTQLPNGRDFHMSV | 3 | 95-110 |
| RVTQLPNGRDFHMS | 58 | 96-109 |
| GWFLDSPDRPWN | 4 | 22-33 |

### Variant Sequences

A sequence variant according to the invention, which has at least 75% identity with one of the first, second, third, and fourth sequences above, preferably has at least 80%, 85%, 90%, 95% or 98% identity with any of the first, second, third, and fourth sequences above.

As understood herein, the percent identity between two peptide sequences may be determined by achieving optimal alignment along the whole length of the sequences, determining the number of aligned positions where the amino-acids are identical in each sequence and by dividing this number by the total number of amino-acids in the longer of the two sequences. The optimal alignment is the one which gives the highest percentage identity between the two sequences.

Also preferably, a sequence variant according to the invention has at least 75%, 80%, 85%, 90%, 95% or 98% identity with SEQ ID NO: 1, SEQ ID NO: 2, SEQ ID NO: 3, SEQ ID NO: 4, or SEQ ID NO: 50, 51, 52, 53, 54, 55, 56, 57, or 58.

The sequence variant according to the invention is such that a polypeptide consisting of the sequence variant must make it possible to elicit an immune response directed against the PD-1 protein; that is to say that the administration of such a peptide, optionally cyclized by the formation of at least one inter-cysteine disulfide bridge, if necessary after adding one or two cysteines within the peptide, and/or at its N-terminal end and/or at its C-terminal end, the peptide being optionally bound to a carrier molecule, in particular a carrier protein, such as KLH (Keyhole Limpet Hemocyanin), to an animal, such as a mouse, a rat or a rabbit, causes the production of antibodies directed against a PD-1 protein, in particular a PD-1 protein of the same species as that to which the sequence with which the sequence variant exhibits the highest percentage identity belongs. The person skilled in the art knows how to determine whether an antibody is directed against PD-1, in particular by carrying out an ELISA test. Preferably, the antibodies elicited by administration of the peptide are blocking or neutralizing, that is to say, they prevent the PD-1 protein from exerting all or part, notably, at least 10%, 25%, 50%, 75%, of its activity, for example measured in vitro. As understood herein, the activity of PD-1 is preferably binding to the PD-L1 protein, which may be measured as in the following Example 2.

### Polypeptide Length

The polypeptide according to the invention preferably comprises at most 200, 150, 100, 90, 80, 70, 60, 50, 40 or 30 amino-acid residues. It is different from the PD-1 protein and does not consist of a portion of more than 30 contiguous amino-acid residues of the PD-1 protein. As the person skilled in the art understands well, this does not exclude that it may consist of two or more portions of the PD-1 protein of at most 30 contiguous amino-acid residues, insofar as these portions are not arranged such as to reconstitute a portion of the PD-1 protein of more than 30 contiguous amino-acids.

As will be clear to those skilled in the art, the polypeptide according to the invention may comprise several repeats, for example 2, 3, 4, 5, 10 or 20 repeats, respectively of the first, second, third, and fourth sequences and of the sequence variant according to the invention.

### Sequences in Addition to the Sequences of the First, Second, Third, and Fourth Sequences and Sequence Variants

Furthermore, the polypeptide according to the invention may also comprise additional sequences not originating from the PD-1 protein.

These additional sequences may in particular provide physicochemical characteristics allowing for improved structural presentation or improved solubility of the polypeptide according to the invention compared to a similar polypeptide which would not include these additional sequences.

The additional sequences may also comprise one or more peptide link sequences, that is to say peptide linkers, useful for linking in particular to a carrier molecule. Such peptide linker sequences typically comprise from 1 to 10, especially 4 to 6, amino-acid residues.

Moreover, these sequences not originating from the PD-1 protein, may also include epitopes belonging to other proteins, thereby making it possible to elicit or generate an immune response directed against these other proteins.

In addition, the polypeptide according to the invention may comprise sequences of exogenous T-cell epitope(s), preferably universal, which makes it possible to enhance the immunogenicity of the polypeptide according to the invention.

The polypeptide according to the invention may also comprise at least one sequence of a carrier protein, for example a virus-like particle (VLP), as described in particular in international application WO 05/117983 for TNF.

### Polypeptide Cyclization

The polypeptide according to the invention may be in linear or cyclized form. Preferably, the polypeptide according to the invention is in cyclized form. This cyclization may be of any type known to those skilled in the art.

The choice of the cyclization strategy according to the invention may in particular take into account optimal antigenic presentation of the epitopes contained in the polypeptide according to the invention and relate only to part of the polypeptide (cyclization within the sequence). Thus, as understood herein, when the polypeptide according to the invention is in cyclized form, only part of the polypeptide may be included in a cycle, while the rest of the polypeptide is in linear form.

Depending on the functional groups present in the polypeptide, this cyclization may be carried out in several different ways, such as for example: from its C-terminal end to its N-terminal end, from its N-terminal end to a side chain, from a side chain to its C-terminal end, or between two side chains. Among the various methods of cyclization of polypeptides, it is possible to cite lactamization, lactonization or the formation of a disulfide bridge. In particular, during the formation of an inter-cysteine disulfide bridge, that is to say between the —SH radicals of two cysteines, the cysteines may already be present in the variant sequence according to the invention or in the first, second, third, and fourth sequences according to the invention, or else be added within these sequences, as well as at their N-terminal and/or C-terminal end.

### Post-Translational Modifications, Amino-Acid Analogs

In addition, the polypeptide according to the invention may comprise post-translational modifications, such as glycosylations, methylations, acylations, in particular by fatty acids, or phosphorylations. In particular, the N-terminal end of the polypeptide according to the invention may be acetylated and the C-terminal end may be modified by amidation.

The polypeptide according to the invention may also comprise one or more analogs or derivatives of amino-acids, including non-natural or non-standard amino-acids, in particular norleucine (Nle).

### Carrier Molecule

Also preferably, the polypeptide according to the invention is attached or linked, in particular by a covalent bond, to a carrier molecule, in particular a carrier protein.

In particular, the carrier molecule may be the Keyhole Limpet Hemocyanin (KLH) protein, the hepatitis B surface antigen (HBsAg), the bovine serum albumin (BSA), the tetanus toxoid (TT) and the toxoid of diphtheria (DT).

The diphtheria toxoid (DT) according to the invention is preferably selected from the group consisting of CRM 197, CRM 176, CRM 228, CRM 45, CRM 9, CRM 102, CRM 103, and CRM 107.

It is particularly preferred that the carrier molecule is CRM 197.

The binding of the polypeptide according to the invention to a carrier molecule, in particular a carrier protein, may be carried out using a heterobifunctional coupling agent, such as the ester of N-γ-maleimidobutyryl-oxysuccinimide (GMBS) and the sulfo-GMBS derivative, m-maleimidobenzoyl-n-hydroxysuccinimide ester (MBS) and the sulfo-MBS derivative, succinimidyl 4-(N-maleimidomethyl) cyclohexane-1-carboxylate (SMCC), a carbodiimide, bisdiazonium-benzidine (BDB) or glutaraldehyde

When GMBS, MBS or SMCC are used, they are preferably attached to a cysteine (C), which if not present in a first, second, third, or fourth sequence, or a variant sequence according to the invention may be added, in particular at its N-terminal or C-terminal end. Furthermore, when a cysteine is present in a first, second, third, or fourth sequence, according to the invention at an undesired position, it is possible to implement, instead, a sequence variant, wherein the cysteine is substituted by another amino-acid, such as a serine.

When using BDB, it is preferably attached to a tyrosine (Y), which if not present in a first, second, third, or fourth sequence, or a sequence variant according to the invention may be added, in particular at its N-terminal or C-terminal end. Furthermore, when a tyrosine is present in a first, second, third, or fourth sequence according to the invention, at an undesired position, it is possible to implement instead a sequence variant, wherein the tyrosine is substituted by another amino-acid, such as a phenylalanine (F).

Furthermore, the linking of the polypeptide according to the invention to a carrier molecule, in particular a carrier protein, can also be achieved using a peptide linker, which binds to the polypeptide according to the invention on one side to the carrier molecule on other side, optionally via a heterobifunctional coupling agent as defined above. Such peptide linkers typically comprise 1 to 10, in particular 4 to 6 amino-acid residues.

Most preferably, the polypeptide according to the invention is attached to the CRM197 carrier protein according to a structure represented by a formula selected from the group consisting of the following formulas:

| | SEQ |
|---|---|
| | ID |
| Construction | NO: |
| CRM197-[GMB-CGAISLAPKAQIKES + Amide]ₙ | 1 |
| [Acetyl + SGAISLAPKAQIKESC-GMB]ₙ-CRM197, | 26 |
| [Acetyl + NWYRMSPSNQTDKLAC-GMB]ₙ-CRM197, | 27 |
| CRM197-[GMB-CNWYRMSPSNQTDKLA + Amide]ₙ, | 28 |
| CRM197-[GMB-CFRVTQLPNGRDFHMSV + Amide]ₙ, | 29 |
| [Acetyl + FRVTQLPNGRDFHMSVC-GMB]ₙ-CRM197 | 30 |
| CRM197-[GMB-cyclo(CGAISLAPKAQIKES)]ₙ | 1 |
| CRM197-[GMB-cyclo(CNWYRMSPSNQTDKLA)]ₙ | 28 |
| CRM197-[GMB-cyclo(CFRVTQLPNGRDFHMSV)]ₙ | 29 |
| CRM197-[GMB-C-cyclo(QGAISLAPKAQIKEK)]ₙ | 31 |
| [cyclo(QAISLAPKAQIKEK)-C-GMB]ₙ-CRM197 | 32 |
| CRM197-[GMB-C-cyclo(QWYRMSPSNQTDKLK)]ₙ | 33 |
| [cyclo(QWYRMSPSNQTDKLK)-C-GMB]ₙ-CRM197 | 34 |
| CRM197-[GMB-C-cyclo(QFRVTQLP | 35 |
| NGRDFHMSVK)]ₙ | |
| [cyclo(QFRVTQLPNGRDFHMSVK)-C- | 36 |
| GMB]ₙ-CRM197 | |
| [cycloS-S(CAISLAPKAQIKEC)-Nle-C- | 37 |
| GMB]ₙ-CRM197 | |
| [cycloS-S(CWYRMSPSNQTDKLC)-Nle-C- | 38 |
| GMB]ₙ-CRM197 | |
| [cycloS-S(CFRVTQLPNGRDFHMSVC)-Nle- | 39 |
| C-GMB]ₙ-CRM197 | |
| [cycloS-S(Acetyl + CGAISLAPKAQIKEC)-G- | 40 |
| Nle-EC + Amide-GMB]ₙ-CRM197 | |
| [cycloS-S(Acetyl + CAISLAPKAQIKC)- | 41 |
| G-Nle-EEC + Amide-GMB]ₙ-CRM197 | |
| [cycloS-S(Acetyl + CWYRMSPSNQTDKLAC)- | 42 |
| GE-Nle-EC-GMB]ₙ-CRM197 | |
| [cycloS-S(Acetyl + CRVTQLPNGRDFHMSC)- | 43 |
| EE-Nle-GC + Amide-GMB]ₙ-CRM197 | |
| CRM197-[GMB-CGWFLDSPDRPWNE + Amide]ₙ | 44 |
| [Acetyl + GWFLDSPDRPWNEC-GMB]ₙ-CRM197 | 45 |
| cyclo(GWFLDSPDRPWN)-EC | 46 |
| [cyclo(QGWFLDSPDRPWNEK)-C-GMB]ₙ-CRM197 | 47 |
| CRM197-[GMB-C-cyclo(QGWFLDSPDRPWNEK)]ₙ | 48 |
| [cycloS-S(CGWFLDSPDRPWNC)- | 49 |
| E-Nle-EC-GMB]ₙ-CRM197 | |

where:
CRM197 denotes the carrier protein,
GMB denotes N-γ-maleimidobutyryl,
Acetyl+ indicates that the N-terminal end is acetylated,
Amide+ indicates that the C-terminal end is modified by amidation,
cyclo( ) indicates lactam-type cyclization between the side chains of C-terminal and N-terminal amino-acid residues,
cycloS-S( ) indicates a disulfide bridge cyclization by disulfide bridge between the sulfhydryl groups of the cysteines present at C-terminal and N-terminal,
the square brackets ([X]n) indicate that one or more polypeptides are attached to the carrier protein and
the underlined part represents the polypeptide according to the invention, the SEQ ID NO of which is shown in the right column.

Thus, most preferably, the polypeptide according to the invention consists of a sequence selected from the group consisting of SEQ ID NO: 1 and SEQ ID NO.:26 to 49.

### Preparation of the Polypeptide

The polypeptide according to the invention may be prepared by any known prior-art method and in particular by chemical synthesis. It is also possible to prepare it through the expression of the nucleic acid according to the invention in eukaryotic or prokaryotic cells.

### Polypeptide Activity

The polypeptide according to the invention, if necessary linked to a carrier molecule, is immunogenic, that is to say that it can elicit or provoke immune response, in particular of the humoral type, that is to say the production of antibodies in a subject, in particular of the mammalian type, to whom it is administered. In particular, the polypeptide according to the invention makes it possible to elicit an immune response directed against the PD-1 protein, in particular anti-PD-1 antibodies, preferably blocking or neutralizing anti-PD-1 antibodies, that is, that is to say that they prevent the PD-1 protein from exercising all or part, in particular at least 10%, 25%, 50%, 75%, of its activity, for example measured in vitro. As understood herein the activity of PD-1 is preferably binding to the PD-L1 protein, which may be measured as shown in Example 2 below.

### Nucleic Acid

The nucleic acid according to the invention is RNA or DNA, preferably DNA. It is preferred that the nucleic acid according to the invention is operably bound to a prokaryotic and/or eukaryotic promoter sequence, especially of the mammalian or viral type. Furthermore, the nucleic acid according to the invention may be included in a vector, such as a plasm id or a virus.

### Antibodies, Antibody Fragments and Aptamers

The antibodies, antibody fragments, and aptamers according to the invention are said to be specifically directed against a polypeptide as defined above when they show essentially no binding to another polypeptide, which does not comprise the polypeptide defined above, under conditions that allow binding of the antibodies, antibody fragments, and aptamers according to the invention, to the polypeptides against which they are specifically directed.

The antibody according to the invention may be polyclonal or monoclonal, preferably monoclonal. Moreover, as understood herein, the “antibody fragments” comprise at least one antigen-binding portion of the antibody from which they are derived, and are in particular of the type Fab, Fab′, F(ab′)₂, Fv stabilized by disulfide (dsFv), dimerized (diabody), trimerized, tetramerized or pentamerized region V, single chain Fv (scFv), region determining complementarity (CDR).

The antibodies may be of any species, in particular human, mouse, rat, rabbit or camelid. Furthermore, when they are non-human, they may be humanized, that is to say that the constant parts of these antibodies are partially or completely replaced by the corresponding constant human parts.

The antibodies according to the invention may be obtained by immunizing an animal with a polypeptide according to the invention according to prior-art methods well known to those skilled in the art.

As understood herein, aptamers are nucleic acids, especially RNAs, capable of specifically binding to a molecular target, such as a protein. The aptamers may in particular be obtained by implementing the SELEX method well known to those skilled in the art from the polypeptides according to the invention.

### Therapeutic Use

### Diseases

Preferably, the disease related to or caused by the expression of the PD-1 protein or of the PD-L1 protein according to the invention, is a cancer or an infectious disease.

More preferably, the disease related to or caused by the PD-1 protein or the PD-L1 protein is selected from the group consisting of:
unresectable metastatic melanoma, advanced metastatic non-small cell lung carcinoma (NSCLC), progressing advanced metastatic NSCLC in particular with or after platinum-based chemotherapy, advanced renal carcinoma, especially metastatic, and locally advanced or metastatic urothelial carcinoma, in particular unresponsive to chemotherapy based on platinum derivatives, prostate cancer, breast cancer, colorectal cancer, or any other cancer where the PD-1/PD-L1 axis is involved in the suppression of an anti-tumor immune response,
bacterial infections, such as pneumonia, meningitis, toxic shock syndrome, food poisoning, gastritis, ulcers, gonorrhea, boils, abscesses, impetigo, ear infections, angina, infections of the urinary and genital tract and bronchopulmonary infections,
viral infections, such as influenza, measles, hepatitis B, hepatitis C, human immunodeficiency virus (HIV) infections, herpes-like viral infections, such as cytomegalovirus or Epstein-Barr virus, herpes, and human papillomavirus (HPV) infections, and
fungal infections, such as blastomycosis, coccidioiodomycosis, histoplamosis, paracoccidioiodomycosis, candidiasis, cryptococcosis, aspergillosis, mucomycosis and pneumocystosis.

### Subject

The subject(s) according to the invention are animals, preferably mammals or marsupials, and more preferably humans, horses, bovines, porcines, sheep, goats, camels, dromedaries, dogs or cats, most preferably humans. It is preferred according to the invention that the polypeptide according to the invention be derived from a PD-1 protein belonging to the same species as the subject in whom the polypeptide is to be used or administered.

### Administration

Preferably, the polypeptide, the pharmaceutical composition, the medication or the product according to the invention is administered or in a form which may be administered orally, mucosally, in particular sublingually, parenterally, intraperitoneally, transcutaneously, intradermally, subcutaneously, intramuscularly, intravenously or intra-arterially.

### Doses

Within the scope of the invention, the polypeptide according to the invention may be administered at doses ranging for example from 1 ng to 1 g, preferably from 1 μg to 1 mg.

### Pharmaceutically Acceptable Vehicle

As intended herein, a “pharmaceutically acceptable vehicle” includes all the compounds, in particular the excipients, which may be administered to a subject in conjunction with a pharmacological active ingredient.

### Adjuvant

Moreover, in particular when used in a vaccine or prophylactic context, the polypeptide according to the invention may be associated to or combined with an adjuvant, or the pharmaceutical composition, the medicament or the product according to the invention may include an adjuvant. The adjuvant may be of any type suitable for increasing the immune response of a subject, animal or human, to the administration of a polypeptide. It may notably be a complete or incomplete Freund adjuvant, Montanide ISA 51 VG, aluminum or aluminum phosphate hydroxide or calcium phosphate, for example, Montanide ISA 51 VG and aluminum or aluminum phosphate hydroxides being preferred. The adjuvant may be associated to the polypeptide according to the invention by producing a 1/1 by volume mixture of an adjuvant solution and of a solution comprising the polypeptide.

### Other Therapy

As understood here, the term “other therapy” refers to a pharmacological therapy with at least one other compound different from the polypeptide according to the invention or a non-pharmacological therapy, such as for example radiotherapy, in particular anti-cancer radiotherapy.

### Other Compound

The other compound useful for eliciting an immune response directed against the PD-1 protein according to the invention may in particular be a polypeptide different from that of the invention derived from the PD-1 protein or a polypeptide derived from PD-L1.

Furthermore, the other compound to be used for the prevention or treatment of a disease linked to or due to the PD-1 protein or to the PD-L1 protein, of a cancer or an infectious disease, may be an anticancer chemotherapy compound, an anticancer immunotherapy compound, for example a monoclonal antibody, an antibiotic, an antiviral, in particular of the interferon type, or an antimycotic.

In addition, in particular when it is used as part of a vaccine or when included in a vaccine or a vaccine composition, the polypeptide according to the invention may be combined with other antigens intended to elicit an immune response against a target different from the PD-1 protein, for example the PD-L1 protein. This type of combination is useful for the preparation of multivalent vaccines.

As used herein, the term “in combination” or “combination product” means that the polypeptide as defined above and the other compound as defined above may be associated within the same pharmaceutical composition or medicament, and therefore may be administered together, or administered separately, that is to say using separate routes of administration and/or separate administration regimens, provided that when administered separately, the periods of prophylactic or therapeutic activity of the polypeptide as defined above and of the other compound as defined above overlap completely or partially.

Thus, when the polypeptide and the other compound are administered separately, the polypeptide as defined above will preferably be administered within 24 hours, more preferably within 2 hours, and even more preferably within 1 hour, following the administration of the other compound as defined above, and its administration will optionally be continued over the following days. Conversely, the other compound as defined above will preferably be administered within 24 hours, more preferably within two hours, and even more preferably within one hour, following administration of the polypeptide, as defined above, and its administration will optionally be continued over the following days. In another preferred embodiment of the invention, when the polypeptide as defined above and the other compound as defined above are administered separately, they are administered essentially simultaneously.

The invention is further illustrated with the following non-limiting figures and Examples.

### DESCRIPTION OF THE FIGURES

FIG. 1 shows the relative amount of anti-huPD-1-antibodies present in the serum diluted at 1/500th of SWISS mice immunized with the PPV-10-01, PPV-10-02, PPV-10-03, PPV-10-04 and PPV-10-05 peptides measured by ELISA (y-axis, optical density (OD) at 450 nm). The horizontal line at 0.2 OD units represents the significance threshold.

FIG. 2 represents the percentage of neutralization of the PD1/PD-L1 interaction obtained with purified antibodies from rabbits (n=4/group) immunized with the peptides PPV-10-01, PPV-10-02, PPV-10-03, PPV-10-04 and PPV-10-05.

### EXAMPLES

### Example 1: Recognition of the Entire Human PD-1 Protein (hPD-1) by the Sera of Mice Immunized with Peptides Derived from hPD-1

Four peptides derived from the human PD-1 protein were produced by solid phase chemical synthesis, then cyclized by adding cysteines to the ends of the epitope of interest and forming of a disulfide bond. They were then coupled via a peptide linker comprising a C-terminal cysteine to a carrier protein, CRM197 (C-Reactive Material 197), using the GMBS coupling agent.

For each conjugate, SWISS mice (Janvier Labs, Le Genest-Saint-Isle, France) free from specific pathogenic organisms were immunized subcutaneously with 100 μg of peptide equivalent derived from human PD-1 (PPV-10-01, PPV-10-02, PPV-10-03, PPV-10-04 or PPPV-10-05; see Table 1) emulsified with Montanide ISA 51 VG adjuvant (n=8 by conjugate). The mice received four subcutaneous injections spaced 15 days apart (D0, D15, D30 and D45).

TABLE 1
Peptides derived from human
PD-1 used for immunization

| | PD-1 | |
|---|---|---|
| Polypeptide | Residues | Structure |
| PPV-10-01 | 123-136 | [cycloS-S(Acetyl + CGAISLAPKA |
| | | QIKEC)-G-Nle-EC + Amide-GMB]ₙ- |
| | | CRM197 |
| PPV-10-02 | 125-135 | [cycloS-S(Acetyl + CAISLAPKAQ |
| | | IKC)-G-Nle-EEC + Amide-GMB]ₙ- |
| | | CRM197 |
| PPV-10-03 | 67-80 | [cycloS-S(Acetyl + CWYRMSPSNQ |
| | | TDKLAC)-GE-Nle-EC-GMB]ₙ- |
| | | CRM197 |
| PPV-10-04 | 96-109 | [cycloS-S(Ac-CRVTQLPNGRDFH |
| | | MSC)EE-Nle-GC + Amide-GMB]- |
| | | CRM197 |
| PPV-10-05 | 22-33 | [cycloS-S(CGWFLDSPDR PWNC)- |
| | | E-Nle-EC-GMB]ₙ-CRM197 |

The amino-acid residues are annotated from the sequence of the human PD-1 protein (SwissProt Q15116)

The relative amount of anti-PD-1 antibody is evaluated in the mouse sera on D54 by ELISA (dilution at 1/500th).

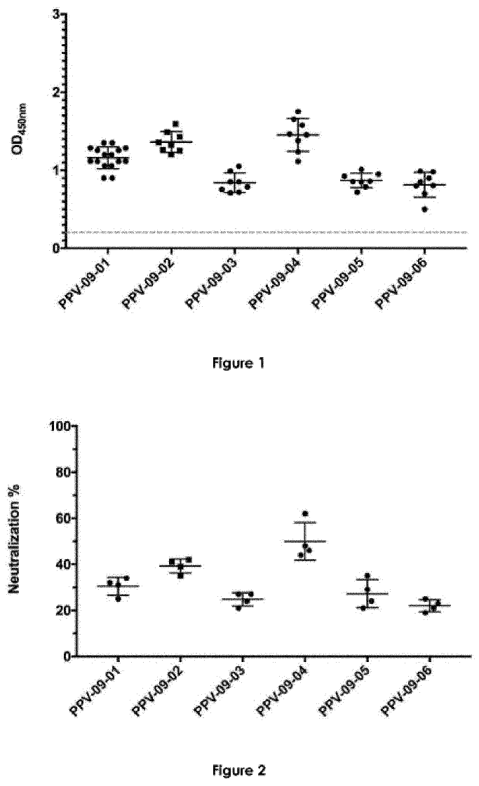

All of the conjugates tested are found to generate the production of antibodies recognizing the human PD-1 protein (FIG. 1).

### Example 2: Neutralization of the Biological Activity of Human PD-1 by Antibodies Purified from the Serum of Rabbits Immunized with Peptides Derived from Human PD-1

The neutralizing capacity of IgGs purified from rabbit serum (n=4/group) immunized with the peptides PPV-10-01, PPV-10-02, PPV-10-03, PPV-10-04 and PPV-10-05, respectively, was evaluated in a cell neutralization assay for PD-1/PD-L1 interaction (Promega, J1250). This test is based on the interaction between 2 cell lines:
a Jurkat effector cell line expressing the human PD-1 gene and the luciferase gene under the control of the NFAT-RE response element,
a CHO—K1 cell line expressing the human PD-L1 gene and a surface protein capable of activating the TCRs in an antigen-dependent fashion.

When the 2 lines are co-cultured, interaction of the PD-1 protein with the PD-L1 protein inhibits signaling via the TCR and luciferase expression (no luminescence will be emitted). In contrast, the addition of anti-PD-1 antibodies neutralizing the interaction of PD-1 with PD-L1 will raise the inhibitory signal, causing the activation of the TCR and the emission of luminescence.

### Description of the Experiment Carried Out:

Plating (D1): inoculation of a 96-well, flat-bottomed plate, treated for cell culture with CHO—K1 cells. Incubating the plate for 20 hours at 37° C.

Incubating the samples and detection (D2): Distribution on the antibody samples to be tested, as well as Jurkat effector cells, on the plate containing the CHO—K1 cells. The plate is then incubated for 6 hours at 37° C.

Addition of the Bio-Glo™ detection reagent in each well. Incubation for 30 minutes at room temperature. Luminescence measurement using a luminometer.

It can be observed that the IgGs of rabbits immunized with the peptides PPV-10-01, PPV-10-02, PPV-10-03, PPV-10-04 and PPV-10-05 neutralize the interaction of the PD-1 protein with the PD-L1 protein at varying degrees, from about 30% to about 60% (FIG. 2).

1. A polypeptide comprising, or consisting of:
a first sequence consisting of at least 8 contiguous amino-acid residues selected from the sequence spanning the amino-acid residues 123 to 137 of the PD-1 protein and at most 30 contiguous amino-acid residues selected from the complete sequence of the PD-1 protein, or a sequence variant having at least 75% identity with the first sequence; and/or
a second sequence consisting of at least 8 contiguous amino-acid residues selected from among the sequence spanning the amino-acid residues 66 to 81 of the PD-1 protein and at most 30 contiguous amino-acid residues selected from the complete sequence of the PD-1 protein, or a sequence variant having at least 75% identity with the second sequence; and/or
a third sequence consisting of at least 8 contiguous amino-acid residues selected from the sequence spanning the amino-acid residues 95 to 110 of the PD-1 protein and at most 30 contiguous amino-acid residues selected from the complete sequence of the PD-1 protein, or a sequence variant having at least 75% identity with the third sequence; and/or
a fourth sequence consisting of at least 8 contiguous amino-acid residues selected from the sequence spanning amino acid residues 22 to 33 of the PD-1 protein and at most 30 contiguous amino-acid residues selected from the complete sequence of the PD-1 protein, or a sequence variant having at least 75% identity with the fourth sequence; and/or
provided that the polypeptide is different from the PD-1 protein and that it does not consist of a portion of more than 30 contiguous amino-acid residues of the PD-1 protein, and
provided that polypeptides respectively consisting of sequence variants of the first, second, third, and fourth sequences make it possible to elicit an immune response directed against the PD-1 protein.

2. The polypeptide according to claim 1, wherein the first sequence, the second sequence, the third and the fourth sequence consist of at least 12 contiguous amino-acid residues respectively selected from the sequences spanning the amino-acid residues 66 to 81, 95 to 110, 123 to 137, and 22 to 33 of the PD-1 protein.

3. The polypeptide according to claim 1, wherein the first sequence, the second sequence, the third and the fourth sequence respectively consist of at most amino-66 to 81, 95 to 110, 123 to 137 and 22 to 33, respectively, of the PD-1 protein.

4. The polypeptide according claim 1, wherein the PD-1 protein is selected from the group consisting of human PD-1 protein, mouse PD-1 protein, monkey PD-1 protein, horse PD-1 protein, bovine PD-1 protein, pig PD-1 protein, sheep PD-1 protein, goat PD-1 protein, camel PD-1 protein and dromedary PD-1 protein, dog PD-1 protein, and cat PD-1 protein.

5. The polypeptide according to claim 1, wherein the PD-1 protein is human PD-1 protein.

6. The polypeptide according to claim 1, wherein:
the first sequence consists of SEQ ID NO: 1, 50, 51, 52, or 54,
the second sequence consists of SEQ ID NO: 2, 55, 56 or 57, and
the third sequence consists of SEQ ID NO: 3, or 58,
the fourth sequence consists of SEQ ID NO: 4.

7. The polypeptide according to claim 1, wherein the polypeptide is in cyclized form.

8. The polypeptide according to claim 1, wherein the polypeptide is linked to a carrier molecule.

9. A nucleic acid encoding a polypeptide as defined in claim 1, or a complement thereof.

10. A pharmaceutical composition comprising, as active substance:
at least one polypeptide as defined in claim 1, or
at least one nucleic acid encoding the at least one polypeptide of claim 1 or a complement of the encoding nucleic acid optionally, in combination with at least one pharmaceutically acceptable carrier.

11. (canceled)

12. An anti-PD-L1 antibody, antibody fragment, or an aptamer specifically directed against the polypeptide as defined in claim 1, provided that the polypeptide does not contain more than two amino-acid residues in addition to the first, to the second, to the third or to the fourth sequence or to their respective sequence variants.

13. (canceled)

14. A method for eliciting an immune response directed against the protein PD-1 in a subject, comprising administering the polypeptide of claim 1, a nucleic acid encoding the polypeptide of claim 1 or a complement of the encoding nucleic acid, or a pharmaceutical composition thereof.

15. A method of preventing or treating a disease linked to or due to the expression of the PD-1 protein or the PD-L1 protein in a subject, comprising administering the polypeptide of claim 1, a nucleic acid encoding the polypeptide of claim 1 or a complement of the encoding nucleic acid.

16. The method of claim 15, wherein the disease is a cancer or infectious disease related to or due to the expression of the PD-1 protein or the PD-L1 protein.

17. The method of claim 15, wherein the disease related to or due to expression of the PD-1 protein or the PD-L1 protein, is selected from the group consisting of:
unresectable metastatic melanoma, advanced metastatic non-small cell lung carcinoma (NSCLC), advanced metastatic NSCLC progressing in particular with or after platinum-based chemotherapy, advanced renal carcinoma, particularly metastatic, and locally advanced or metastatic urothelial carcinoma, particularly unresponsive to chemotherapy based on platinum derivatives, prostate cancer, breast cancer, colorectal cancer, or any other cancer, where the PD-1-PD-L1 axis is involved in the suppression of an anti-tumor immune response.
bacterial infections, such as pneumonia, meningitis, toxic shock syndrome, food poisoning, gastritis, ulcers, gonorrhea, boils, abscesses, impetigo, ear infections, angina, infections of the urinary and genital tract and bronchopulmonary infections,
viral infections, such as influenza, measles, hepatitis B, hepatitis C, human immunodeficiency virus (HIV) infections, herpes-like viral infections, such as cytomegalovirus or Epstein-Barr virus, herpes, and human papillomavirus (HPV) infections, and
fungal infections, such as blastomycosis, coccidioiodomycosis, histoplamosis, paracoccidioiodomycosis, candidiasis, cryptococcosis, aspergillosis, mucomycosis and pneumocystosis.
